(19) United States
(12) Patent Application Publication
(10) Pub. No.: US 2025/0075923 A1
(43) Pub. Date: Mar. 6, 2025

(54) HYBRID UV-C UPPER AIR TREATMENT DEVICE

(71) Applicant: Ralph Gregory Greene, Dalton, GA (US)

(72) Inventor: Ralph Gregory Greene, Dalton, GA (US)

(21) Appl. No.: 18/817,367

(22) Filed: Aug. 28, 2024

Related U.S. Application Data
(60) Provisional application No. 63/579,485, filed on Aug. 29, 2023.

(51) Int. Cl. F24F 8/22; A61L 9/20

(52) U.S. Cl. CPC F24F 8/22; A61L 9/20

(57) ABSTRACT

A hybrid UV-C upper air treatment device may include a treatment chamber formed in a housing. One or more air inlets may be in communication with the treatment chamber, and one or more air outlets may be in communication with the treatment chamber. A fan assembly may be coupled to the housing, and the fan assembly may be configured to draw air into the treatment chamber through the air inlets and to cause the air to exit the treatment chamber through the air outlets. A UV-C light emitting element may be coupled to the housing, and the UV-C light emitting element may be configured to generate UV-C light. The UV-C light may be communicated into the treatment chamber and a portion of the UV-C light in the treatment chamber may be configured to exit the device through one or more of the air inlets.

### CROSS REFERENCE TO RELATED APPLICATIONS

This application claims priority to and the benefit of the filing date of U.S. Provisional Application No. 63/579,485, filed on Aug. 29, 2023, entitled “Hybrid UV-C Upper Air treatment device”, which is hereby incorporated by reference in its entirety.

### FIELD OF THE INVENTION

This patent specification relates to the field of UV-C upper air treatment devices and methods. More specifically, this patent specification relates to a ceiling mounted hybrid device which is able to provide improved UV-C upper air treatment.

BACKGROUND

In the past several years, the need for air sanitization and air treatment in work and public spaces has become very urgent with the Covid-19 Virus outbreak, but it is also important for protection against other infectious viruses, bacteria, and molds.

There are various types of Air Treatment devices such as HEPA Filtration and Ultraviolet Light that are used to inactivate or trap airborne infectious bacteria, viruses, and molds. Ultraviolet-C (UV-C) light is a type of ultraviolet light that is used to kill germs and neutralize microorganisms. It's also known as germicidal irradiation (UVGI). UV-C light is shorter than visible light and is part of the electromagnetic spectrum, which is categorized by wavelength. UVC light typically has a wavelength of 180-280 nanometers.

There are generally 2 types of UV-C air treatment devices, one is an “upper air device” which shines UV-C light into the upper part of a room and has baffles to help keep the UV-C light which can be harmful to eyes, skin and some materials from going into occupied parts of the room below a set height, for example seven feet. This type of UV-C air treatment device is simple in design and shines UV-C light externally from the device into an upper part of a room and does not utilize fans to move air through the device or around the room.

Another type of UV-C air treatment device is an “enclosed” UV-C air treatment device which typically has a fan or fans to pull in or move air to run the air past the UV-C lamp to treat the air and then sends the air out through a vent. This type of device generally does not have UV-C Light outside the device and has less risk of exposure for people and materials.

However, both of these existing types of devices have a relatively low cleaned air flow rate (CFM) which results in greater required run times and decreased efficiency. The higher the cleaned air flow rate (CFM) created by the device, the faster a room can be effectively treated, and the risk of airborne infectious bacteria, viruses and mold can be reduced.

Therefore, a need exists for a novel device which is able to provide improved UV-C upper air treatment and which are able to provide improved functionality over existing devices.

### BRIEF SUMMARY OF THE INVENTION

A hybrid UV-C upper air treatment device is provided. In preferred embodiments, the device may use a single UV-C light emitting element for both Upper Air and enclosed UV-C air treatment. The current invention preferably may comprise an under ceiling mounted device which greatly increases the UV-C effective dosage and efficacy for in-activating airborne bacteria, viruses and mold in a room by: directing UV-C light into the upper part of a room; by pulling the air into the device with one or more fan assemblies; directing the air to move close to the UV-C light emitting element inside the device; and then directing the air through an air outlet vent in a downward flow into the room to help reduce infectious airborne cross flow contaminations.

In some embodiments, the device may include a housing, and a treatment chamber may be formed in the housing. An air inlet may be coupled to the housing, and the air inlet may be in fluid communication with the treatment chamber. An air outlet may be coupled to the housing, and the air outlet is in fluid communication with the treatment chamber. A fan assembly may be coupled to the housing. The fan assembly may be configured to draw air into the treatment chamber through the air inlet, and the fan assembly may be configured to cause the air to exit the treatment chamber through the air outlet. A UV-C light emitting element may be coupled to the housing, and the UV-C light emitting element may be configured to generate UV-C light. The UV-C light may be communicated into the treatment chamber, and a portion of the UV-C light in the treatment chamber may be configured to exit the device through the air inlet.

In further embodiments, the device may include a housing, and a treatment chamber may be formed in the housing. A first air inlet may be coupled to the housing, and the first air inlet may be in fluid communication with the treatment chamber. A second air inlet may be coupled to the housing. The second air inlet may be in fluid communication with the treatment chamber, and the second air inlet may be coupled to the housing so that the first air inlet and the second air inlet are positioned on opposite sides of the housing. An air outlet may be coupled to the housing, and the air outlet may be in fluid communication with the treatment chamber. A fan assembly may be coupled to the housing, and the fan assembly may be configured to draw air into the treatment chamber through the first air inlet and the second air inlet. The fan assembly may also be configured to cause the air to exit the treatment chamber through the air outlet. The air drawn into the treatment chamber through the first air inlet may enter the first air inlet in a first direction, and the air exiting the treatment chamber through the air outlet may exit the air outlet in a second direction. The air drawn into the treatment chamber through the second air inlet may enter the second air inlet in a third direction, and the second direction may be substantially perpendicular to both the first direction and the third direction. A UV-C light emitting element may be coupled to the housing, and the UV-C light emitting element may be configured to generate UV-C light. The UV-C light may be communicated into the treatment chamber. A first portion of the UV-C light in the treatment chamber may be configured to exit the device through the first air inlet, and a second portion of the UV-C light in the treatment chamber may be configured to exit the device through the second air inlet.

Numerous objects, features and advantages of the present invention will be readily apparent to those of ordinary skill in the art. Some example objects of the present invention are listed below.

One object of the present invention is to provide a hybrid UV-C upper air treatment device that allows the use of standard and existing suspended grid ceiling light fixtures such as LED Flat Panel Lights, LED Troffers, etc. to be repositioned to a set lower level to provide a space for a housing that can incorporate an air treatment system. An advantage to utilizing an existing light fixture position is that the light fixture and the air treatment system can use the same incoming power lines so that no new power lines need to be added which can be a major expense. A further advantage is that the current invention can utilize the existing or a standard size and model ceiling light fixture.

Another object is to provide a hybrid UV-C upper air treatment device in which external upper air is treated with UV-C and air is also treated in the device internally and the air is moved with fans to increase circulation inside the room and directs the UV-C treated air flow horizontally and downward into the room.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts a top perspective view of an example of a hybrid UV-C upper air treatment device according to various embodiments described herein.

FIG. 2 illustrates a top plan view of an example of a hybrid UV-C upper air treatment device according to various embodiments described herein.

FIG. 3 shows a bottom plan view of an example of a hybrid UV-C upper air treatment device according to various embodiments described herein.

FIG. 4 depicts a first side view of an example of a hybrid UV-C upper air treatment device according to various embodiments described herein.

FIG. 5 illustrates a second side view of an example of a hybrid UV-C upper air treatment device according to various embodiments described herein.

FIG. 6 shows a third side view of an example of a hybrid UV-C upper air treatment device according to various embodiments described herein.

FIG. 7 depicts a fourth side view of an example of a hybrid UV-C upper air treatment device according to various embodiments described herein.

FIG. 8 illustrates a sectional, through line 8-8 shown in FIG. 6, plan view of an example of a hybrid UV-C upper air treatment device according to various embodiments described herein.

FIG. 9 shows a sectional, through line 9-9 shown in FIG. 4, elevation view of an example of a hybrid UV-C upper air treatment device according to various embodiments described herein.

FIG. 10 depicts a top perspective view of another example of a hybrid UV-C upper air treatment device according to various embodiments described herein.

FIG. 11 shows a block diagram depicting some example components that a hybrid UV-C upper air treatment device may include according to various embodiments described herein.

FIG. 12 depicts a block diagram illustrating an example of a control unit according to various embodiments described herein.

FIG. 13 depicts a side elevation view of an example hybrid UV-C upper air treatment device coupled to a ceiling according to various embodiments described herein.

FIG. 14 depicts a side elevation view of an example hybrid UV-C upper air treatment device coupled to a wall according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

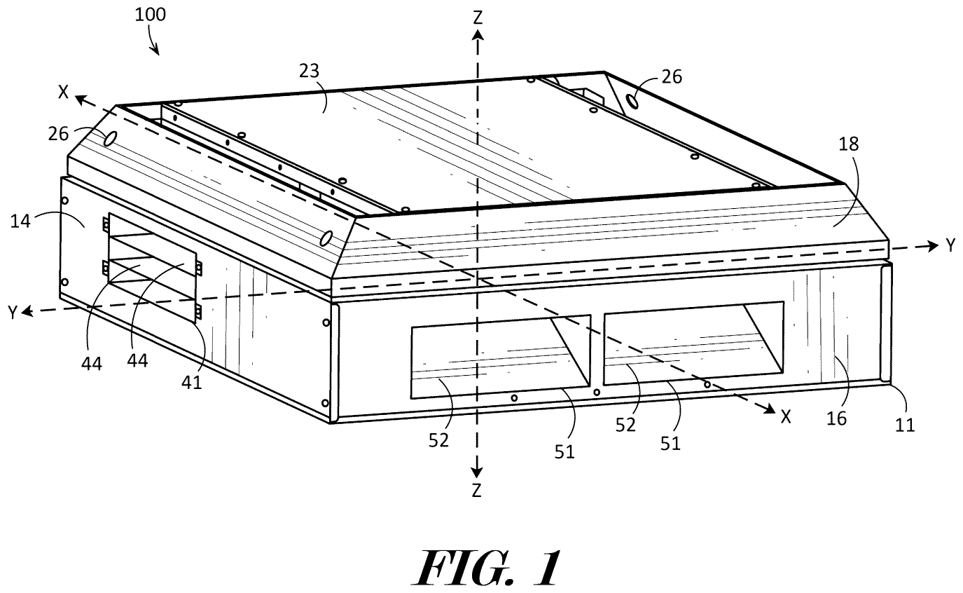

For purposes of description herein, the terms “upper,” “lower,” “left,” “right,” “rear,” “front,” “side,” “vertical,” “horizontal,” and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Although the terms “first,” “second,” etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 20% of the specified number. Additionally, as used in this application, the term “substantially” means that the actual value is within about 10% of the actual desired value, more preferably within about 5% of the actual desired value and even more preferably within about 1% of the actual desired value of any variable, element or limit set forth herein.

A new hybrid UV-C upper air treatment device is discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

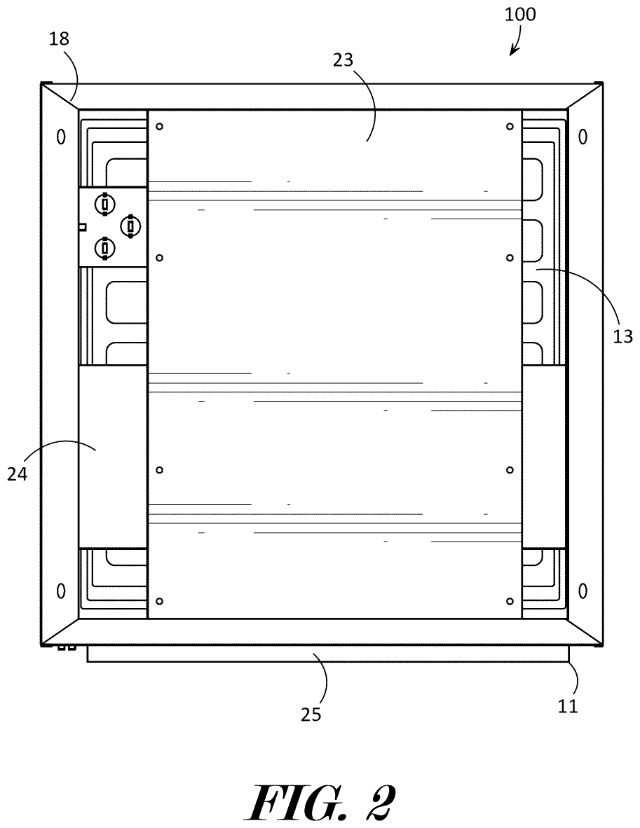

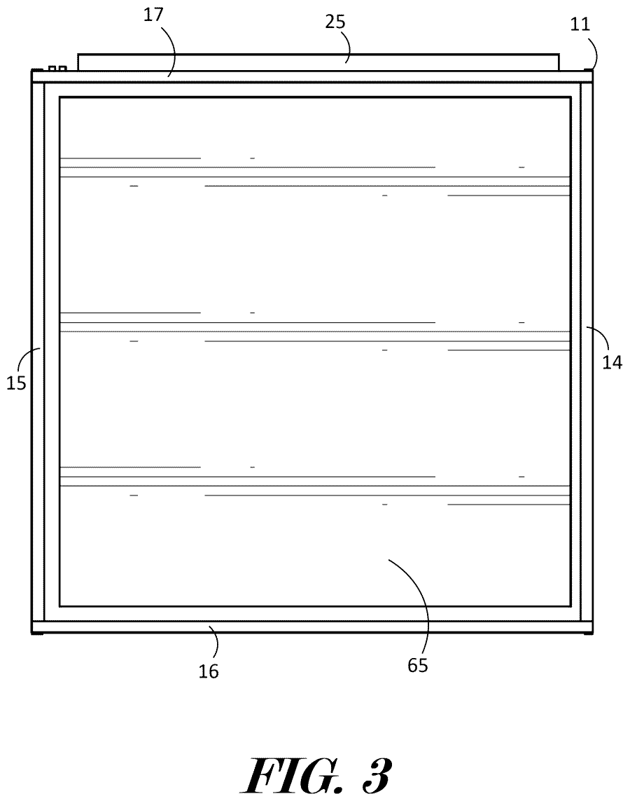

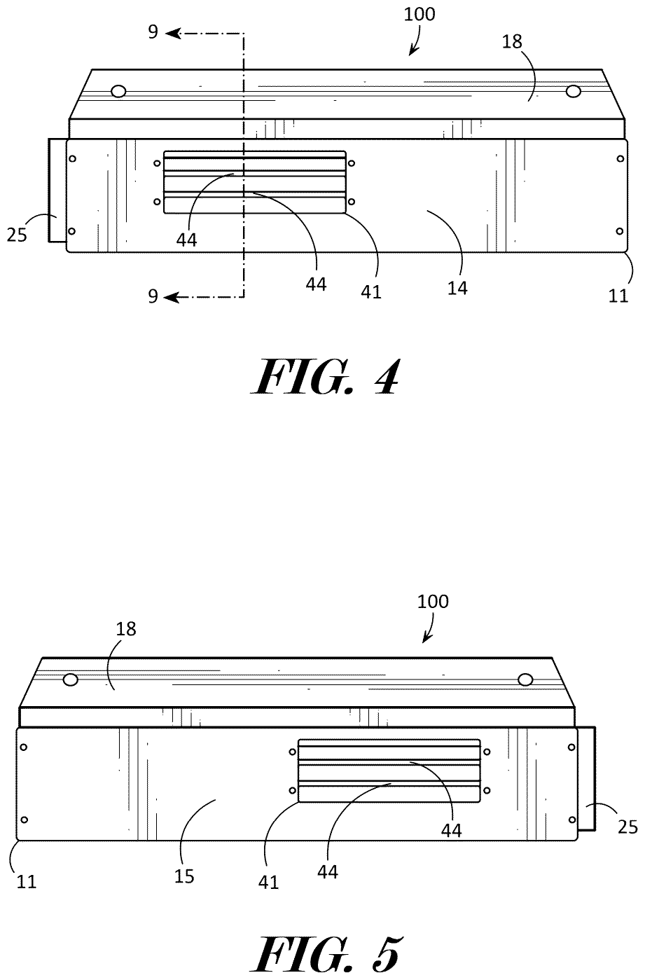

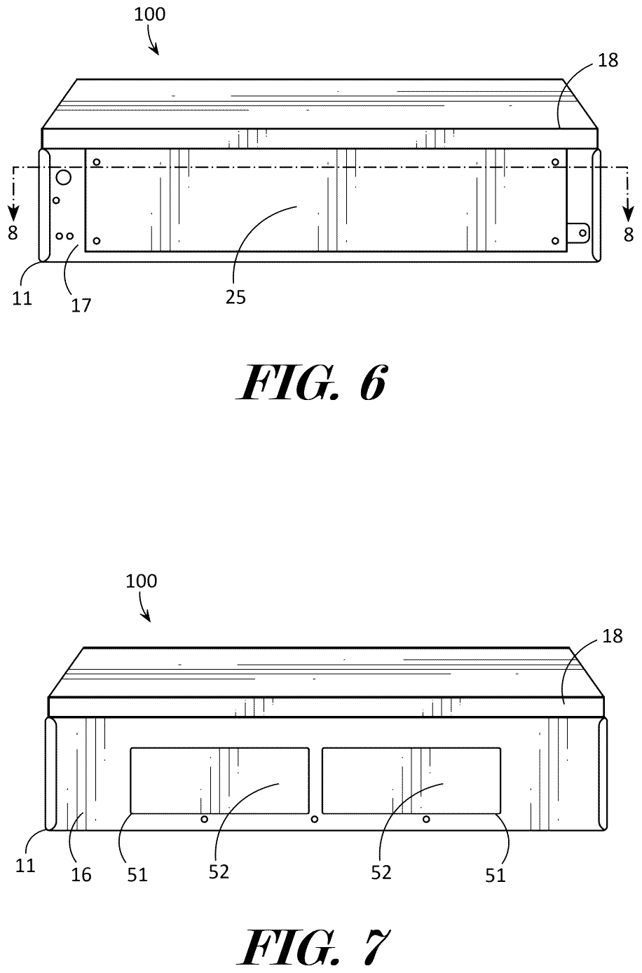

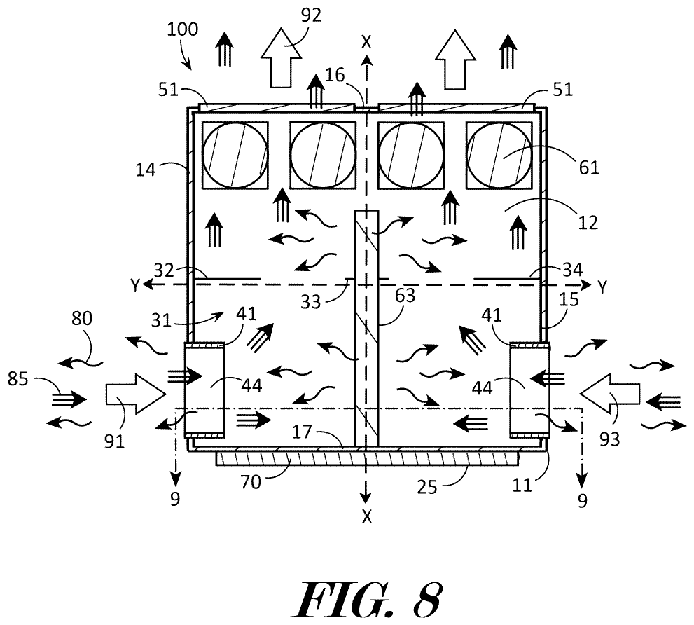

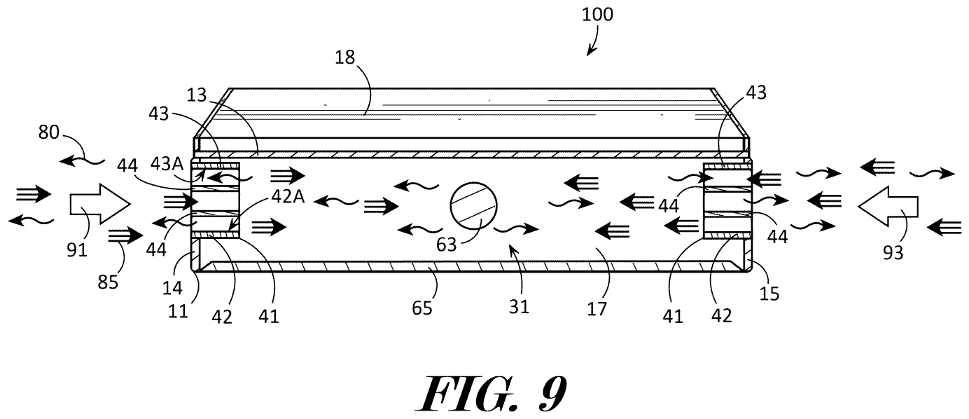

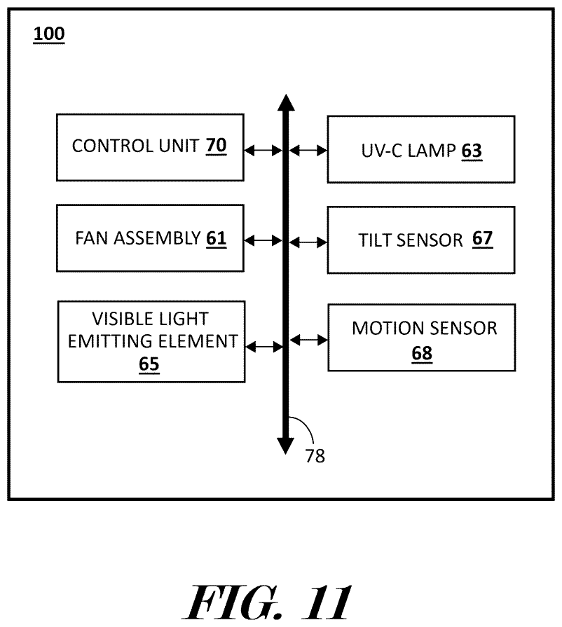

The present invention will now be described by example and through referencing the appended figures representing preferred and alternative embodiments. FIGS. 1-11 illustrate examples of a hybrid UV-C upper air treatment device (“the device”) 100 according to various embodiments. In some embodiments, the device 100 may comprise a housing 11, and a treatment chamber 31 may be formed in the housing 11. One or more air inlets 41 may be coupled to the housing 11, and the air inlets 41 may be in communication with the treatment chamber 11. One or more air outlets 51 may be coupled to the housing 11, and the air outlets 51 may be in communication with the treatment chamber 31. A fan assembly 61 may be coupled to the housing 11. The fan assembly 61 may be configured to draw air 85 (shown with arrows having three tails in FIGS. 8 and 9) into the treatment chamber 31 through the air inlets 41, and the fan assembly 61 is configured to cause the air 85 to exit the treatment chamber 31 through the air outlets 51. A UV-C light emitting element 63 may be coupled to the housing 11, and the UV-C light emitting element 63 may be configured to generate UV-C light 80 (shown with arrows having wavy tails in FIGS. 8 and 9). The UV-C light 80 may be communicated into the treatment chamber 31, and a portion of the UV-C light 80 in the treatment chamber 31 may be configured to exit the device 100 through one or more of the air inlets 41.

The device 100 may comprise a housing 11 that may be configured in any size and shape. The housing 11 may comprise one or more walls, such as a bottom wall 12, a top wall 13, a first side wall 14, a second side wall 15, a first end wall 16, and a second end wall 17. The walls 12, 13, 14, 15, 16, 17, may be configured in any size and shape to form a housing 11 of any size and shape. In preferred embodiments, the housing 11 may comprise a generally rectangular prism shape having generally rectangular shaped side walls 14, 15, that may be opposingly positioned to each other and which may be coupled to generally rectangular shaped end walls 16, 17, which may be opposingly positioned to each other. The side walls 14, 15, and end walls 16, 17, may be coupled to the bottom wall 12 and the top wall 13 with the bottom wall 12 and the top wall 13 opposingly positioned to each other.

Optionally, the housing 11 may comprise one or more support walls 23, 24, which may be coupled to one or more walls 12, 13, 14, 15, 16, 17, for structural reinforcement and other fabrication purposes. Optionally, the housing 11 may comprise an electrical access cover 25 which may provide a quick release covering for electronics of the device 100 and preferably to allow user access to a treatment chamber 31 for cleaning purposes and UV-C light emitting element 63 and fan assembly 61 replacement and service.

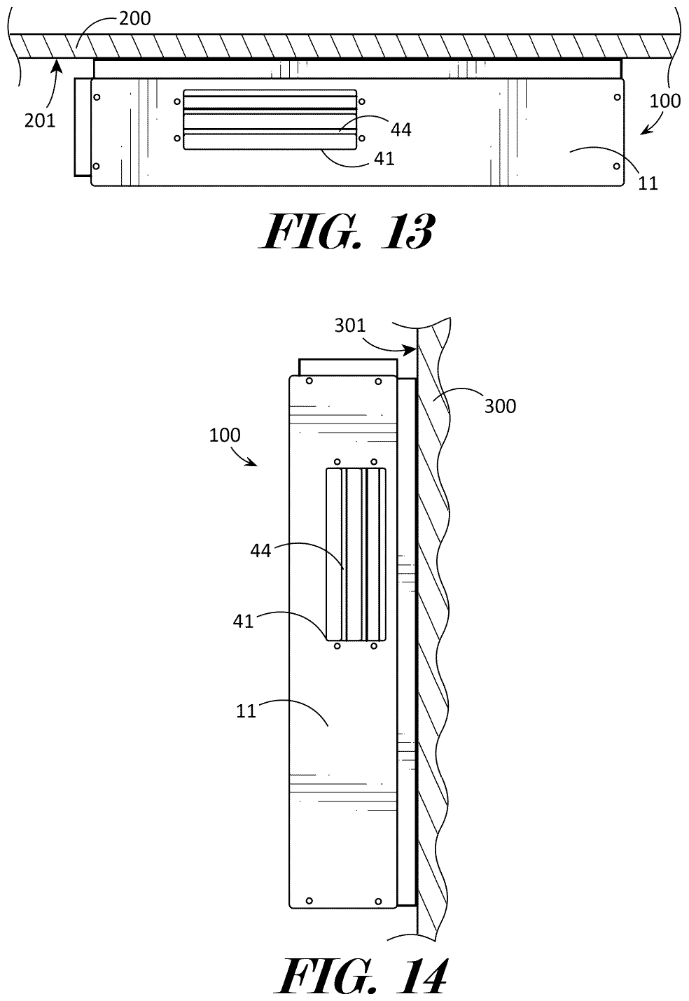

In preferred embodiments, the housing 11 may be coupled to a ceiling 200 (FIG. 13), ceiling surface 201 (surface of the ceiling 200 that faces the room or space that people may occupy) (FIG. 13), or other overhead structure so that the bottom wall 12 faces down and towards individuals that are below the device 100 and so that the top wall 13 faces up and away from individuals that are below the device 100. Preferably, the housing 11 may comprise a grid support frame 18 which may be coupled to or proximate to the top wall 13 and/or which may be coupled to one or more of the side walls 14, 15, and end walls 16, 17. Generally, a grid support frame 18 may be used to couple the device 100 to a suspended ceiling support grid or to another structure or coupling method in order to couple the device 100 to a ceiling or other overhead structure so that the bottom wall 12 faces down and towards individuals that are below the device 100. Preferably, a grid support frame 18 may comprise one or more frame apertures 26 which may be used to receive fasteners, hanger supports 19, and other coupling devices or methods that may be used to couple the device 100 to a suspended ceiling support grid or to another structure or coupling method in order to couple the device 100 to a ceiling 200 or other overhead structure so that the bottom wall 12 faces down and towards individuals that are below the device 100. In further embodiments, the housing 11 may be couple to a wall 300 (FIG. 14), wall surface 301 (surface of a wall that faces the room or space that people may occupy) (FIG. 14), or other surface or structure that may be below a ceiling 200.

The device 100 may comprise a treatment chamber 31 that may be formed in the housing 11. A treatment chamber 31 may comprise a cavity that air 85 passing through the device 100 must travel through after entering the device 100 through and air inlet 41 and before exiting the device 100 through and air outlet 51. A treatment chamber 31 may be configured in any size and shape. Preferably, the interior surfaces of one or more walls 12, 13, 14, 15, 16, 17, may form surfaces of the treatment chamber 31 and may be made from or may comprise a UV reflective material, such as a reflective metal surface, reflective paint or other coating, etc.

The device 100 may comprise a UV-C light emitting element 63 that may be coupled to the housing 11, and the UV-C light emitting element 63 may be configured to generate UV-C light 80 so that the UV-C light emitting element 63 may provide Ultraviolet germicidal irradiation (UVGI). A UV-C light emitting element 63 should be understood to include any source of man-made UV light generating technology including lamps, LEDs, and types such as 2-pin, 4-pin, screw-in, T5 bi-pin, plug-in compact, etc. In preferred embodiments, a UV-C light emitting element 63 may be configured to generate UV-C light 80 that comprises or that is substantially Ultraviolet C (UV-C) radiation with a wavelength of approximately 100-280 nanometers. In some embodiments, a UV-C light emitting element 63 may comprise one or more UV-C generating LEDs that may be in a enclosed strip to form an elongated assembly similar to a UVC lamp or can be in smaller strips or blocks and placed in multiple locations within the treatment chamber 31 to provide increased UV-C dosage within specific areas of the device 100.

The device 100 may comprise a UV-C light emitting element 63 that may be coupled to the housing 11 so that the UV-C light 80 generated by the UV-C light emitting element 63 may be communicated into the treatment chamber 31. In preferred embodiments, UV-C light 80 generated by a UV-C light emitting element 63 may be communicated into the treatment chamber 31 by at least a portion of the UV-C light emitting element 63 being positioned within the treatment chamber 31. In further embodiments, a UV-C light emitting element 63 may be positioned outside the treatment chamber 31 and the UV-C light 80 generated by a UV-C light emitting element 63 may be communicated into the treatment chamber 31 by a transparent wall the forms a portion of the treatment chamber 31, by a mirror or other reflective device that is able to direct UV-C light 80 generated by a UV-C light emitting element 63 into the treatment chamber 31, or via any other suitable UV-C light 80 conducting or directing device or method.

The device 100 may comprise one or more fan assemblies 61 that may be coupled to the housing 11 and that may be configured to draw air 85 into the treatment chamber 31 through an air inlet 41 and to cause the air 85 to exit the treatment chamber 31 through an air outlet 51. Preferably, a fan assembly 61 may comprise a rotating arrangement of vanes or blades which may act on air 85 within the treatment chamber 31 to cause or direct the air 85 to flow and to proceed into the air inlets 41 and out of the air outlets 51. A fan assembly 61 may comprise or may be in communication with an electric motor, such as a brushed DC motor, brushless DC motor, switched reluctance motor, universal motor, AC polyphase squirrel-cage or wound-rotor induction motor, AC SCIM split-phase capacitor-start motor, AC SCIM split-phase capacitor-run motor, AC SCIM split-phase auxiliary start winding motor, AC induction shaded-pole motor, wound-rotor synchronous motor, hysteresis motor, synchronous reluctance motor, pancake or axial rotor motor, stepper motor, etc., which may be used to move the vanes or blades of the fan assembly 61. Example fan assemblies 61 include axial-flow fans, centrifugal fans, cross-flow fans, bellows, Coandă effect air movers, electrostatic air movers, or any other device or method capable of moving air 85.

The device 100 may comprise one or more air inlets 41 which may be coupled to the housing 11 and which may be in fluid communication with the treatment chamber 31 to enable air 85 that is exterior to the device 100 to enter the device 100 through the one or more air inlets 41 and travel into a treatment chamber 31. An air inlet 41 may comprise one or more openings, apertures, conduits, etc. which may enable air 85 to be communicated through the housing 11 and into a treatment chamber 31 and which may enable UV light 80 within the treatment chamber 31 to exit the device 100 through the air inlet 41. Generally, an air inlet 41 may be in communication with the treatment chamber 31 and may enable fluid communication for air 85 to enter a treatment chamber 31 and, optionally, for at least a portion of UV light 80 within the treatment chamber 31 to exit the device 100 through the air inlet 41.

Air inlets 41 may be configured in any size and shape. Preferably, an air inlet 41 may comprise a lower inlet wall 42, having a lower inlet wall surface 42A, and an upper inlet wall 43, having an upper inlet wall surface 43A, and the lower inlet wall 42 may be positioned relatively closer to the bottom wall 12 while the upper inlet wall 43 may be positioned relatively closer to the top wall 13. Air 85 entering the treatment chamber 31 via the air inlet 41 and UV-C light 80 optionally exiting the treatment chamber 31 via the air inlet 41 may pass between the lower inlet wall surface 42A of the lower inlet wall 42 and the upper inlet wall surface 43A of the upper inlet wall 43. In preferred embodiments, the lower inlet wall 42 may comprise a flat or planar shaped lower inlet wall surface 42A (as perhaps best shown in FIG. 9) and the upper inlet wall 43 may comprise a flat or planar shaped upper inlet wall surface 43A (as perhaps best shown in FIG. 9), and the flat or planar shaped lower inlet wall surface 42A and the flat or planar shaped upper inlet wall surface 43A may be substantially parallel (plus or minus 10 degrees) to each other as perhaps best shown in FIG. 9.

In some embodiments, an air inlet 41 may comprise one or more, such as a plurality of, light guide plates 44 that may be positioned between a lower inlet wall 42 and an upper inlet wall 43. Light guide plates 44 of an air inlet 41 may be in communication with the treatment chamber 31 so that air 85 entering the treatment chamber 31 via the air inlet 41 and UV-C light 80 optionally exiting the treatment chamber 31 via the air inlet 41 may pass across, such as above and below, light guide plates 44 of the air inlet 41.

Preferably, light guide plates 44 of an air inlet 41 may direct or limit the UV light 80 emitted by a UV-C light emitting element 63 in a horizontal direction extends generally parallel to a floor surface that the device 100 is positioned above and/or a ceiling surface 201 that the device 100 is coupled to or below. In some embodiments, the one or more light guide plates 44 may direct or limit the UV light 80 emitted by a UV-C light emitting element 63 in a horizontal direction that is above the bottom wall 12 and a visible light emitting element 65 that may be coupled to the bottom wall 12 and/or that is below the top wall 13. In some embodiments, light guide plates 44 of the air inlet 41 may be spaced apart at a minimum of between approximately 0.1 inches and 10.0 inches apart, and more preferably between 0.5 inches and 3.0 inches apart, from each other.

In preferred embodiments, light guide plates 44 may be made from or may comprise a material that is resistant to UV-C light 80 and/or may be coated with a material coating that may be resistant to UV-C light 80. In preferred embodiments, light guide plates 44 may be made from or may comprise a material that is anti-reflective to UV-C light 80 wavelengths, such as carbon black, ceramics (typically ceramics may be metal oxides, though some ceramics may be nitrides, borides and carbides that exhibit strong covalent bonding). Example materials and coatings that are resistant to UV-C light 80 may include UV light resistant paints and coatings, UV light resistant materials, such as aluminum, zinc coated steel, stainless steel, other metals and metal alloys, chrome coating, ceramics, etc. In some embodiments, light guide plates 44 may be made from or may comprise a material that is generally reflective to UV-C light 80 wavelengths. Example materials and coatings that are reflective to UV-C light 80 wavelengths include aluminum, zinc coated steel, other metals and metal alloys, chrome coating, etc.

Light guide plates 44 may be configured in any size and shape. Preferably, an air inlet 41 may comprise a plurality of light guide plates 44 that may be generally flat or planar in shape. A flat or planar in shape light guide plate 44 may have a height dimension (extending towards the lower inlet wall 42 and upper inlet wall 43) that may be substantially less (such as less than ten percent) than the length and/or width of the light guide plate 44 as perhaps best shown in FIG. 9. In further preferred embodiments, two or more, such as each, light guide plate 44 of an air inlet 41 may be planar in shape and may be substantially parallel (plus or minus ten degrees) to each other as perhaps best shown in FIG. 9. In preferred embodiments, the lower inlet wall 42 may comprise a flat or planar shaped lower inlet wall surface 42A (as perhaps best shown in FIG. 9) and the upper inlet wall 43 may comprise a flat or planar shaped upper inlet wall surface 43A (as perhaps best shown in FIG. 9), and the flat or planar shaped lower inlet wall surface 42A and the flat or planar shaped upper inlet wall surface 43A may be substantially parallel (plus or minus 10 degrees) to one or more flat or planar in shape light guide plates 44 as perhaps best shown in FIG. 9.

In some embodiments, the device 100 may comprise one or more UV-C light emitting elements 63 that may be coupled to the housing 11 and UV-C light 80 generated by at least one UV-C light emitting element 63 may be communicated into the treatment chamber 31, and one or more portions of the UV-C light 80 in the treatment chamber 31 may be configured to exit the device 100 through one or more air inlets 41. For example, the device 100 may comprise one or more UV-C light emitting elements 63 with UV-C light 80 generated by at least one UV-C light emitting element 63 communicated into the treatment chamber 31, and a first portion of the UV-C light 80 in the treatment chamber 31 may be configured to exit the device 100 through a first air inlet 41 and a second portion of the UV-C light 80 in the treatment chamber 31 may be configured to exit the device 100 through a second air inlet 41.

In some embodiments, the device 100 may comprise a UV-C light emitting element 63 that may be coupled to the housing 11, and all or at least a portion of the UV-C light emitting element 63 may be positioned within the treatment chamber so that the UV-C light emitting element 63 generates UV-C light 80 within the treatment chamber 31, and one or more portions of the UV-C light 80 in the treatment chamber 31 may be configured to exit the device 100 through one or more air inlets 41. For example, the device 100 may comprise a single UV-C light emitting element 63 with at least a portion of the UV-C light emitting element 63 positioned within the treatment chamber 31 so that UV-C light 80 generated by the UV-C light emitting element 63 is communicated into the treatment chamber 31, and a first portion of the UV-C light 80 in the treatment chamber 31 may be configured to exit the device 100 through a first air inlet 41 and a second portion of the UV-C light 80 in the treatment chamber 31 may be configured to exit the device 100 through a second air inlet 41.

The device 100 may comprise one or more air outlets 51 which may be coupled to the housing 11 and which may be in fluid communication with the treatment chamber 31 to enable air 85 that is in the treatment chamber 31 to exit the treatment chamber 31 and the device 100 via the one or more air outlets 51. An air outlet 51 may comprise one or more openings, apertures, conduits, etc. which may enable air 85 to be communicated through the housing 11 and out of a treatment chamber 31. Preferably, a fan assembly 61, baffles 52, treatment chamber interior walls 32, 33, 34, or other element may prevent UV-C light 80 within the treatment chamber 31 from exiting the device 100 through an air outlet 51. In some embodiments, the device 100 may comprise a baffle 52 that may be coupled to or proximate to an air outlet 51 and which may be configured to direct air 85 exiting the air outlet 51 in a horizontal direction 92 that is away from the device 100 and optionally also in a downward vertical direction, such as generally toward a ground or floor surface that is below the device 100.

In some embodiments, air 85 that is drawn into the treatment chamber 31 through the one or more air inlets 41 may enter the air inlets 41 in a different direction than air 85 that exits the one or more air outlets 51. To aid in the discussion of directions of air 85 entering and exiting the device 100, the device 100 may comprise an x-axis (shown by line X-X in FIGS. 1 and 8), a y-axis (shown by line Y-Y in FIGS. 1 and 8), and a z-axis (shown by line Z-Z in FIG. 1) in which the x-axis and y-axis denote horizontal directions and which the z-axis denotes vertical directions (toward and away from a ceiling 200 and floor. In preferred embodiments, air 85 that is drawn into the treatment chamber 31 through the one or more air inlets 41 may enter the air inlets 41 in a generally horizontal direction 91, 93, and air that exits the one or more air outlets 51 may exit the air outlets 51 in a different generally horizontal direction 92 that is different than the generally horizontal direction(s) 91, 93, of air entering the air inlets 41.

As a first example, and referring to FIGS. 8 and 9, the device 100 may comprise an air inlet 41 coupled side wall 14 and an air outlet 51 coupled to a second end wall 17. Air 85 drawn into the treatment chamber 31 through the air inlet 41 may enter the air inlet 41 in a first direction 91 (preferably in a first horizontal direction substantially parallel (plus or minus 20 degrees) with y-axis), air 85 exiting the treatment chamber 31 through the air outlet 51 may exit the air outlet 51 in a second direction 92 (preferably in a second horizontal direction substantially parallel (plus or minus 20 degrees) with x-axis), and the second direction 92 may be substantially perpendicular (plus or minus 20 degrees) to the first direction 91.

As a second example, and still referring to FIGS. 8 and 9, the device 100 may comprise two air inlets 41 coupled to opposing side walls 14, 15, and two air outlets 51 coupled to a second end wall 17. Air 85 drawn into the treatment chamber 31 through a first air inlet 41 may enter the first air inlet 41 in a first direction 91 (preferably in a first horizontal direction substantially parallel (plus or minus 20 degrees) with y-axis), air 85 exiting the treatment chamber 31 through the air outlets 51 may exit the air outlets 51 in a second direction 92 (preferably in a second horizontal direction substantially parallel (plus or minus 20 degrees) with x-axis), air 85 drawn into the treatment chamber 31 through a second air inlet 41 may enter the second air inlet 41 in a third direction 93 (preferably in a third horizontal direction substantially parallel (plus or minus 20 degrees) with y-axis), and the second direction 92 may be substantially perpendicular (plus or minus 20 degrees) to both the first direction 91 and the third direction 93. Preferably, the first 91 and third 93 directions may be substantially parallel to each other.

In some embodiments, the device 100 may comprise a visible light emitting element 65 that is configured to illuminate an environment external to the housing 11, such as below the housing 11 when the device 100 is coupled to a ceiling 200 or other structure that is above a floor surface. In preferred embodiments, a visible light emitting element 65 may generate light between 400 and 700 nanometers (nm) in wavelength, such as that is commonly used in indoor environments. A visible light emitting element 65 may comprise any suitable visible light emitting device, such as a light-emitting diode (LED), incandescent light bulb, fluorescent light bulb, halogen light bulb, high-intensity discharge light bulb, electroluminescent light source, neon light source, or any other type of suitable light source. In preferred embodiments, a visible light emitting element 65 may comprise standard size two feet by two feet, two feet by four feet, etc., LED Flat panel light fixtures, Troffer light fixtures, and other light fixtures. A Troffer light fixture is a rectangular light fixture that fits into a modular dropped ceiling grid (i.e. two feet by two feet or two feet by four feet). Troffer fixtures typically accommodate standard fluorescent lamps (T12, T8, or T5), integral LED sources, and other light sources.

Suspended Grid type ceilings 200 are widely used in office buildings and in spaces. The grid and tile panels predominantly come in two sizes: two feet by two feet or two feet by four feet. Support bars or “Tee” sections make up the interior grids of the Grid ceilings and are suspended by hanger wires that support and suspend the loaded weight of the ceiling grid and light fixtures. To provide lighting with a suspended grid ceiling, there are several types of light fixtures that are made and sized to insert into a grid in the place of a tile panel. Fluorescent lights are one type, LED edge lite, and LED Backlit panels are other examples. These lighting fixtures are typically called “Panel Lights” as they are made to replace a panel in the suspended grid ceiling. The Panel light is held up and supported by the bottom leg of the support bars or “Tee” sections. There are many types, makes and models of these Panel lights which are produced in large quantities and are economical due to the large scale of production. In preferred embodiments, a visible light emitting element 65 may comprise a Panel light.

In some embodiments, the housing 11 of the device 100 may be coupled directly to a ceiling 200, such as a suspended grid ceiling utilizing the suspended grid ceiling's support bars or “Tee” sections as a support for the main housing 11. To accomplish this, the device 100 may comprise a grid support frame 18 and housing 11 that may be sized and shaped to fit inside the same dimensions as a typical Panel tile or a typical Panel light. Preferably, the grid support frame 18 may be made to the same outside dimensions as a panel tile or panel light fixture so that the grid support frame 18 fits into the same space and is supported by the ceiling's support bars or “Tee” sections. In preferred embodiments, a grid support frame 18 may comprise a four-sided frame made out of metal angle material, but in further embodiments a grid support frame 18 may be configured in various forms, such a square tubing or even a solid sheet.

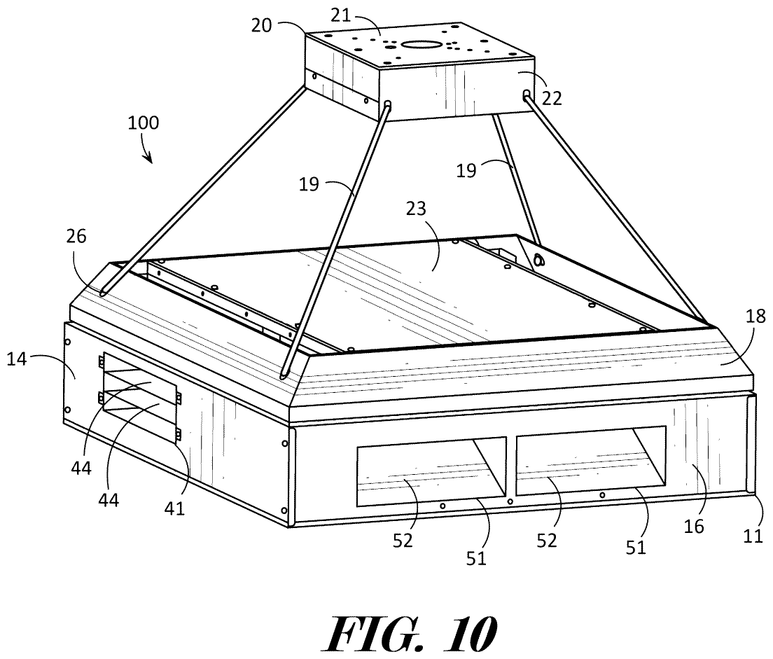

In some embodiments, the housing 11 of the device 100 may be coupled indirectly to a ceiling 200, such as by all or portions of the housing 11 being suspended below the ceiling 200. Optionally, and as shown in FIG. 10, the device 100 may comprise one or more hanger supports 19 and a ceiling mounting box 20 which may be used to couple the housing 11 and optionally a grid support frame 18 to a ceiling 200.

Hanger supports 19 may comprise metal wire, cable, or other elongated material capable of supporting all or a portion of the weight of the device 100. Hanger supports 19 may be coupled to any element of the housing 11 and/or a grid support frame 18 via any suitable coupling method. A ceiling mounting box 20 may comprise a mounting plate 21 that may be used to couple the device 100 to a ceiling 200 or other structure that is above the location that the device 100 is to be positioned in. A mounting plate 21 may comprise a horizontal plate of metal or other substantially rigid material that may have one or more apertures for receiving screws or other fasteners that may be used to couple the device 100 to a ceiling 200 and/or to an object or structure that is coupled to a ceiling 200. One or more vertical walls 22 may be coupled to the mounting plate 21 to form a ceiling mounting box 20. The mounting plate 21 may be coupled to a junction box or ceiling mounting box, as is commonly used in ceiling fan mounting, via a T-grid mounting bracket, as is commonly used in ceiling fan mounting, or the mounting plate 21 may be coupled to a hard or sturdy ceiling 200 as desired. The elongated length of the hanger supports 19 may be selected to suspend the housing 11 a desired distance from the ceiling mounting box 20 and/or a desired distance from one or more elements of the ceiling 200.

In some embodiments, the device 100 may comprise a tilt sensor 67 that may be configured to detect the tilt or orientation of the device 100. A tilt sensor 67 is an instrument that is used for measuring the tilt in one or more axes of a reference plane. Tilt sensors 67 measure the tilting position of objects with reference to gravity and are used in numerous applications. They enable the easy detection of orientation or inclination. Tilt sensors 67 may include mercury switches, tilt switches or rolling ball sensors, a micro electro-mechanical system (MEMS) tilt sensor, any other type of device or sensor which may be used to detect an object being tilted in one or more directions. As an example, a tilt sensor 67 may have a tilt switch that may include a metallic ball that is designed to move when the sensor reaches a predetermined angle, and the metallic ball may make electrical contact when the device 100 is tilted or not tilted to generate electrical communication to indicate tilt or to interrupt electrical communication in response to tilt, e.g., to deactivate motion of the one or more fan assemblies 61 and/or to deactivate a UV-C light emitting element 63.

In preferred embodiments, the device 100 may comprise a tilt sensor 67 that may detect tilt of the device 100 in the x-axis (shown by line X-X) and in the y-axis (shown by line Y-Y), as tilt in the x-axis or y-axis would result in the device 100 being tilted out of the z-axis (shown by line Z-Z) and therefore result in the device 100 being tilted out of vertical. In some embodiments, the device 100 may comprise a tilt sensor 67 that is configured to detect the device 100 being tilted, and the one or more UV-C light emitting elements 63 may be deactivated (so as to stop generating UV-C light 80) if the tilt sensor 67 detects that the device 100 has been tilted to exceed a tilt threshold. In some embodiments, the device 100 may comprise a tilt sensor 67 that is configured to detect the device 100 being tilted, and the one or more fan assemblies 61 may be deactivated (so as to stop moving or motivating air through the treatment chamber 31) if the tilt sensor 67 detects that the device 100 has been tilted to exceed a tilt threshold. In some embodiments, the tilt threshold may be defined by the device 100 being tilted more than ten degrees, such as between ten to thirty or more degrees, out of at least one of the x-axis, y-axis, and z-axis. In some embodiments, the tilt threshold may be defined by the device 100 being tilted more than five degrees out of at least one of the x-axis, y-axis, and z-axis. In preferred embodiments, a tilt sensor 67 and/or a processor 71 of a control unit 70 may deactivate one or more UV-C light emitting elements 63 and/or one or more fan assemblies 61 when the tilt sensor 67 detects that the device 100 has been tilted to exceed a tilt threshold and that the tilt threshold has been exceeded for a time period, such as more than five seconds, more than ten seconds, etc., to prevent deactivation of the one or more UV-C light emitting elements 63 and/or one or more fan assemblies 61 if the device 100 is accidentally bumped or momentarily impacted, swayed, etc.

In some embodiments, the device 100 may comprise a control unit 70 which may be in electronic communication with the one or more fan assemblies 61, one or more UV-C light emitting elements 63, and optional one or more tilt sensors 67.

In some embodiments, a control unit 70 may enable electronic communication between the one or more fan assemblies 61, one or more UV-C light emitting elements 63, and optional one or more tilt sensors 67 so that the one or more tilt sensors 67 may be able to activate and/or deactivate the one or more fan assemblies 61 and/or one or more UV-C light emitting elements 63. For example, a control unit 70 may comprise a circuit board or other wired electronic interface.

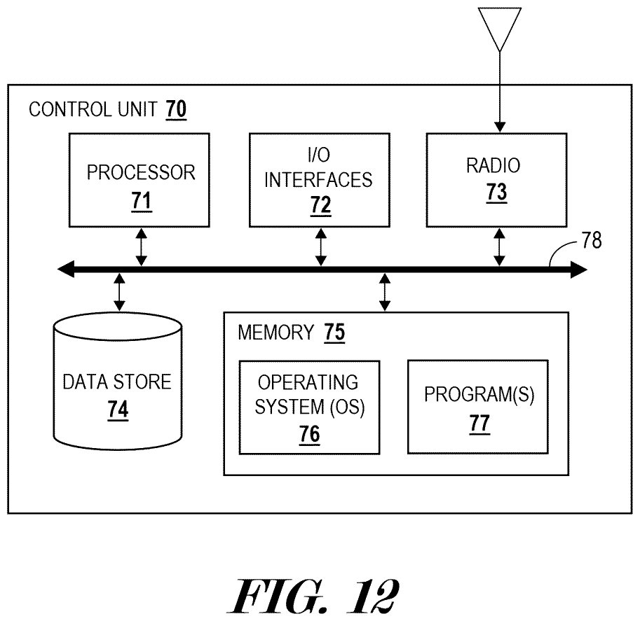

In some embodiments, a control unit 70 may be in electronic communication with the one or more fan assemblies 61, one or more UV-C light emitting elements 63, and optionally one or more tilt sensors 67 and optionally one or more motion sensors 68 so that the control unit 70 may be able to activate and/or deactivate the one or more fan assemblies 61 and/or one or more UV-C light emitting elements 63 based on tilt information communicated to the control unit 70 from a one or more tilt sensors 67. As an example, the device 100 can be a digital device that, in terms of hardware architecture, comprises a control unit 70 which may include one or more of a processor 71, input/output (I/O) interfaces 72, a radio module 73, a data store 74, and memory 75. It should be appreciated by those of ordinary skill in the art that FIG. 12 depicts the control unit 70 in an oversimplified manner , and a practical embodiment may include additional components or elements and suitably configured processing logic to support known or conventional operating features that are not described in detail herein.

In some embodiments, the device 100 may include various sensors and timers to activate or de-activate the device 100, and/or to activate or de-activate one or more fan assemblies 61 and/or UV-C light emitting elements 63 according to a schedule. In some embodiments, the device 100 may include a motion sensor 68 that may be configured to detect motion proximate to the device 100. Example motion sensors 68 include: passive infrared motion sensors, microwave motion sensors, ultrasonic motion sensors, tomographic motion sensors, video camera software, or any other type of sensor configured to detect motion in an area. In preferred embodiments, the device 100 may comprise a motion sensor 68 that may be configured to activate or de-activate the device 100, and/or to activate or de-activate one or more fan assemblies 61 and/or UV-C light emitting elements 63. Motion proximate to the motion sensor 68 may comprise motion being detected within 50 feet of the motion sensor 68, more preferably within 20 feet of the motion sensor 68, and more preferably within 10 feet of the motion sensor 68. In preferred embodiments, motion detected by a motion sensor 68 may be used to activate the device 100 (activate one or more fan assemblies 61 and/or UV-C light emitting elements 63) when people are present in the room and the one or more fan assemblies 61 and/or UV-C light emitting elements 63 may be configured to run a set time after presence is detected to fully sanitize the air in the room which helps to reduce surface contamination.

The control unit 70 and electronic elements (61, 63, 65, 67, 68) may be communicatively coupled via a local interface 78. The local interface 78 can be, for example but not limited to, one or more buses, circuit boards, wiring harnesses, or other wired connections or wireless connections, as is known in the art. The local interface 78 can have additional elements, which are omitted for simplicity, such as controllers, buffers (caches), drivers, repeaters, and receivers, among many others, to enable communications. Further, the local interface 78 may include address, control, and/or data connections to enable appropriate communications among the aforementioned components.

A processor 71 is a hardware device for executing software instructions. A processor 71 can be any custom made or commercially available processor, a central processing unit (CPU), an auxiliary processor among several processors associated with the control unit 70, a semiconductor-based microprocessor (in the form of a microchip or chip set), or generally any device for executing software instructions. When the control unit 70 is in operation, the processor 71 is configured to execute software stored within the memory 75, to communicate data to and from the memory 75, and to generally control operations of the device 100 pursuant to the software instructions and/or from instructions received from a remote control and/or other electronic elements (61, 63, 65, 67, 68). In an exemplary embodiment, the processor 71 may include a mobile optimized processor, such as optimized for power consumption and mobile applications.

The I/O interfaces 72 can be used by a user to provide user input and display system output data, such as operational status, from the device 100. The I/O interfaces 72 can include, for example, buttons, knobs, switches, LED indicator lights, LED display, LCD display, a serial port, a parallel port, a small computer system interface (SCSI), an infrared (IR) interface, a radio frequency (RF) interface, a universal serial bus (USB) interface, and the like. In some embodiments, I/O interfaces 72 may comprise buttons, knobs, switches, etc., that may be manipulated by a user 201 to enable the user to select one or more settings for the motor 45 and UV light emitting elements 31.

A radio module 73 may enable wireless communication to an external access device or network. In preferred embodiments, a radio module 73 may operate via Wi-Fi communication standards. Any number of suitable wireless data communication protocols, techniques, or methodologies can be supported by the radio module 73, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Near-Field Communication (NFC); Frequency Hopping Spread Spectrum; Long Term Evolution (LTE); cellular/wireless/cordless telecommunication protocols (e.g. 3G/4G/5G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication.

An optional data store 74 may be used to store data. The data store 74 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 74 may incorporate electronic, magnetic, optical, and/or other types of storage media.

The memory 75 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 75 may incorporate electronic, magnetic, optical, and/or other types of storage media. Note that the memory 75 may have a distributed architecture, where various components are situated remotely from one another, but can be accessed by the processor 71. The software in memory 75 can include one or more software programs, each of which includes an ordered listing of executable instructions for implementing logical functions. In the example of FIG. 12, the software in the memory system 75 optionally includes a suitable operating system (O/S) 76 and program(s) 77. The operating system 76 essentially controls the execution of input/output interface 72 and other element functions, and provides scheduling, input-output control, file and data management, memory management, and communication control and related services. The operating system 76 may be, for example, LINUX (or another UNIX variant), Android (available from Google), Symbian OS, Microsoft Windows CE, Microsoft Windows 7 Mobile, iOS (available from Apple, Inc.), webOS (available from Hewlett Packard), Blackberry OS (Available from Research in Motion), and the like. The programs 77 may include various applications, add-ons, etc. configured to provide end user functionality of the device 100. In a typical example, one or more of the programs 77 may comprise instructions for controlling the functions of the motor 45 and UV light emitting elements 31.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The control unit 70 may also include a main memory, such as a random access memory (RAM) or other dynamic storage device (e.g., dynamic RAM (DRAM), static RAM (SRAM), and synchronous DRAM (SDRAM)), coupled to the bus for storing information and instructions to be executed by the processor 71. In addition, the main memory may be used for storing temporary variables or other intermediate information during the execution of instructions by the processor 71. The control unit 70 may further include a read only memory (ROM) or other static storage device (e.g., programmable ROM (PROM), erasable PROM (EPROM), and electrically erasable PROM (EEPROM)) coupled to the bus for storing static information and instructions for the processor 71.

While some exemplary shapes and sizes have been provided for elements of the device 100, it should be understood to one of ordinary skill in the art that the housing 11, treatment chamber 31, and any other element described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Additionally, while some materials have been provided, in other embodiments, the elements that comprise the device 100 may be made from or may comprise durable materials such as aluminum, steel, other metals and metal alloys, wood, hard rubbers, hard plastics, fiber reinforced plastics, carbon fiber, fiberglass, resins, polymers or any other suitable materials including combinations of materials. Additionally, one or more elements may be made from or may comprise durable and slightly flexible materials such as soft plastics, silicone, soft rubbers, or any other suitable materials including combinations of materials. In some embodiments, one or more of the elements that comprise the device 100 may be coupled or connected together with heat bonding, chemical bonding, adhesives, clasp type fasteners, clip type fasteners, rivet type fasteners, threaded type fasteners, other types of fasteners, or any other suitable joining method. In other embodiments, one or more of the elements that comprise the device 100 may be coupled or removably connected by being press fit or snap fit together, by one or more fasteners such as hook and loop type or Velcro® fasteners, magnetic type fasteners, threaded type fasteners, sealable tongue and groove fasteners, snap fasteners, clip type fasteners, clasp type fasteners, ratchet type fasteners, a push-to-lock type connection method, a turn-to-lock type connection method, a slide-to-lock type connection method or any other suitable temporary connection method as one reasonably skilled in the art could envision to serve the same function. In further embodiments, one or more of the elements that comprise the device 100 may be coupled by being one of connected to and integrally formed with another element of the device 100.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A hybrid UV-C upper air treatment device, the device comprising:
a housing;
a treatment chamber formed in the housing;
an air inlet coupled to the housing, wherein the air inlet is in fluid communication with the treatment chamber;
an air outlet coupled to the housing, wherein the air outlet is in fluid communication with the treatment chamber;
a fan assembly coupled to the housing, wherein the fan assembly is configured to draw air into the treatment chamber through the air inlet, and wherein the fan assembly is configured to cause the air to exit the treatment chamber through the air outlet; and
a UV-C light emitting element coupled to the housing, the UV-C light emitting element configured to generate UV-C light, wherein the UV-C light is communicated into the treatment chamber, and wherein a portion of the UV-C light in the treatment chamber is configured to exit the device through the air inlet.

2. The device of claim 1, wherein a portion of the UV-C light emitting element is positioned within the treatment chamber so that the UV-C light emitting element generates UV-C light within the treatment chamber, and wherein the portion of UV-C light that exits the device through the air inlet is treated by the UV-C light emitting element.

3. The device of claim 1, wherein the air drawn into the treatment chamber through the air inlet enters the air inlet in a first direction, wherein the air exiting the treatment chamber through the air outlet exits the air outlet in a second direction, and wherein the first direction is substantially perpendicular to the second direction.

4. The device of claim 1, further comprising a visible light emitting element that is configured to illuminate an environment external to the housing with light between 400 and 700 nanometers (nm) in wavelength.

5. The device of claim 1, further comprising a tilt sensor that is configured to detect the device being tilted, wherein the UV-C light emitting element is deactivated if the tilt sensor detects that the device has been tilted to exceed a tilt threshold.

6. The device of claim 1, further comprising a tilt sensor that is configured to detect the device being tilted, wherein the fan assembly and the UV-C light emitting element are both deactivated if the tilt sensor detects that the device has been tilted to exceed a tilt threshold.

7. The device of claim 1, wherein the air inlet comprises an upper inlet wall and a lower inlet wall, and wherein the UV-C light generated within the treatment chamber is configured to exit the device between the upper inlet wall and the lower inlet wall.

8. The device of claim 1, wherein the air inlet comprises a plurality of light guide plates.

9. The device of claim 1, wherein the air inlet comprises a first light guide plate that is planar in shape and a second light guide plate that is planar in shape.

10. The device of claim 9, wherein the first light guide plate is substantially parallel to the second light guide plate.

11. A hybrid UV-C upper air treatment device, the device comprising:
a housing;
a treatment chamber formed in the housing;
a first air inlet coupled to the housing, wherein the first air inlet is in fluid communication with the treatment chamber;
a second air inlet coupled to the housing, wherein the second air inlet is in fluid communication with the treatment chamber, and wherein the second air inlet is coupled to the housing so that the first air inlet and the second air inlet are positioned on opposite sides of the housing;
an air outlet coupled to the housing, wherein the air outlet is in fluid communication with the treatment chamber;
a fan assembly coupled to the housing, wherein the fan assembly is configured to draw air into the treatment chamber through the first air inlet and the second air inlet, wherein the fan assembly is configured to cause the air to exit the treatment chamber through the air outlet, wherein the air drawn into the treatment chamber through the first air inlet enters the first air inlet in a first direction, wherein the air exiting the treatment chamber through the air outlet exits the air outlet in a second direction, wherein the air drawn into the treatment chamber through the second air inlet enters the second air inlet in a third direction, and wherein the second direction is substantially perpendicular to both the first direction and the third direction; and
a UV-C light emitting element coupled to the housing, the UV-C light emitting element configured to generate UV-C light, wherein the UV-C light is communicated into the treatment chamber, and wherein a first portion of the UV-C light in the treatment chamber is configured to exit the device through first air inlet, and wherein a second portion of the UV-C light in the treatment chamber is configured to exit the device through the second air inlet.

12. The device of claim 11, wherein a portion of the UV-C light emitting element is positioned within the treatment chamber so that the UV-C light emitting element generates UV-C light within the treatment chamber, wherein the first portion of UV-C light that exits the device through the first air inlet is generated by the UV-C light emitting element, and wherein the second portion of UV-C light that exits the device through the second air inlet is generated by the UV-C light emitting element.

13. The device of claim 11, wherein the first direction is substantially parallel to the third direction.

14. The device of claim 11, further comprising a visible light emitting element that is configured to illuminate an environment external to the housing with light between 400 and 700 nanometers (nm) in wavelength.

15. The device of claim 11, further comprising a tilt sensor that is configured to detect the device being tilted, wherein the UV-C light emitting element is deactivated if the tilt sensor detects that the device has been tilted to exceed a tilt threshold.

16. The device of claim 11, further comprising a tilt sensor that is configured to detect the device being tilted, wherein the fan assembly is deactivated if the tilt sensor detects that the device has been tilted to exceed a tilt threshold.

17. The device of claim 11, wherein the first air inlet comprises an upper inlet wall and a lower inlet wall, and wherein the UV-C light generated within the treatment chamber is configured to exit the device between the upper inlet wall and the lower inlet wall.

18. The device of claim 11, wherein the first air inlet comprises a plurality of light guide plates.

19. The device of claim 11, wherein the first air inlet comprises a first light guide plate that is planar in shape and a second light guide plate that is planar in shape.

20. The device of claim 19, wherein the first light guide plate is substantially parallel to the second light guide plate.
